(19) United States
(12) Patent Application Publication
(10) Pub. No.: US 2013/0115174 A1
(43) Pub. Date: May 9, 2013

(54) TRANSMUCOSAL COMPOSITION CONTAINING ANTHOCYANINS FOR ALLEVIATING A VISUAL DISCOMFORT

(71) Applicants: Yves Lepelletier, Athis-Mons (FR); Réda Hadj-Slimane, Paris (FR); Tewfik Hadl-Slimane, Relizane (DZ)

(72) Inventors: Yves Lepelletier, Athis-Mons (FR); Réda Hadj-Slimane, Paris (FR); Tewfik Hadl-Slimane, Relizane (DZ)

(21) Appl. No.: 13/810,871

(22) PCT Filed: Jul. 29, 2011
(86) PCT No.: PCT/EP2011/063130

§ 371 Date: Jan. 17, 2013

(30) Foreign Application Priority Data
Jul. 29, 2010 (EP) 10302843.4

(51) Int. Cl. A61K 36/45; A61K 31/522; A61K 31/375

(52) U.S. Cl. CPC A61K 36/45; A61K 31/375; A61K 31/522
USPC 424/48; 424/732; 424/725

(57) ABSTRACT

The present invention relates to a composition comprising a natural extract containing anthocyanins and an agent for enhancing vigilance, and to a method for alleviating visual discomfort related to or caused by oxidative stress comprising administering via transmucosal route the composition of the invention, said composition being in a form adapted to transmucosal administration, said form preferably being a chewing gum, an orodispersible/orodisintegrating tablet or film or a buccal spray.

### FIELD OF INVENTION

The present invention relates to a composition comprising a natural extract containing anthocyanins and an agent for enhancing vigilance, and a method for alleviating visual discomfort related to or caused by oxidative stress. More specifically, the invention relates to a method for alleviating visual discomfort comprising administering via transmucosal route the composition of the invention.

### BACKGROUND OF INVENTION

Oxidative stress corresponds to an excess of oxidant elements as compared to antioxidant products, resulting in structural or formal damages, such as cellular damages. Oxidative stress is linked to an excessive production of reactive oxygen species (ROS) coupled to a deficiency in anti-radical defenses. Several scientific studies have demonstrated that oxidative stress is influenced by genetic factors (Mata et al., Invest Ophthalmol Vis Sci 2001; 42:1685-90), constitutive factors, such as aging, or environmental risks such as tobacco, UV light exposition or hypercholesterolemia (Wihlmark et al., Free radic. Biol. Med. 1997; 22:1229-34; Mares-Perlman et al., Arch Ophthalmol. 1995; 113(12):1518-23).

In retina, the oxidation of lipids linked to amino acids leads to the accumulation of lipofuscine, a fluorescent pigment. Said pigment sensitizes epithelial cells to blue light and induces the production of ROS inducing cell death by apoptosis (Wihlmark et al., Free radic. Biol. Med. 1997; 22:1229-34).

Moreover, with aging, the catalytic activity of catalase, an antioxidant enzyme participating to antioxidant protection is decreased in the retinal epithelium.

Consequently, with aging, oxidative damages in the retina may cause a loss of visual function, such as, for example a loss of visual acuity, a poor night vision, an enhanced light sensitivity and glare.

A poor visual function, together with a high reaction time (which may result from a loss of vigilance as well as from a visual dysfunction) and a state of hypovigilance is one of the major human risk factor for car crash. Developing compositions maintaining visual function, normal reaction time and vigilance of car drivers may be considered as a public road safety stake.

Non-enzymatic antioxidants exist to counterbalance the effect of oxidation, for example in the eye. Said antioxidants are for example carotenoids (lutein, zeaxanthin . . . ) or anthocyanins which belong to the family of flavonoids. Anthocyanins may be formed of anthocyanosides, such as for example delphinidol, cyanidol, malvidol, petunidol glucosides as well as of quinolizidinic bases.

The use of non-enzymatic antioxidant for preventing or treating oxidative stress-related eye disorders or for alleviating visual discomfort related to oxidative stress is well-known in the art. For example, anthocyanins, which can be extracted from natural vegetal species, such as for example bilberry fruits, present antioxidant properties commonly used in compositions for treating or preventing eye disorders. In vitro, anthocyanins protect retinal cells against oxidative stress (Dutot et al., J Fr Ophtalmol., 2008; 31(10):975-80).

WO200577176 discloses the use of anthocyanins for treating or preventing eye diseases such as Age-related Macular Degeneration (AMD), glaucoma, as well as for increasing the resistance of retinal cells to blue-light damages and for limiting lipofuscine accumulation. US2003/0008048 describes a composition to be ingested, comprising anthocyanins, lutein and vitamin C for helping the body resist the effects of aging process. Anthocyanins are used to be administered through an oral way by ingestion, leading to digestive absorption of the substance. However, said absorption is not efficient, as only about 1% of total anthocyanins are systemically absorbed after oral administration of bilberry extracts containing 25% of anthocyanins.

Taking into account this issue, this invention aims at improving the bioavailability of anthocyanins present in natural, preferably vegetal, extracts. This improvement of bioavailability carries the further advantage that, according to the invention, the amount of anthocyanins to be administered to a subject for alleviation of visual discomfort is lower than that to be administered by ingestion route.

Surprisingly, the Applicant herein shows that the combination of Vitamin C or Caffeine, with anthocyanins, improved the bioavailability of anthocyanins and resulted in an improved alleviation of visual discomfort, especially visual discomfort related to oxidative damages, together with an enhancement of the state of vigilance. Thus, the composition of the invention, comprising anthocyanins and an agent for alleviating vigilance has an effect on visual function, reaction time and vigilance. As such, the composition of the invention may be of particular interest for car drivers.

### Definitions

In the present invention, the following terms have the following meanings:

The term “Nutraceutical” refers to a product isolated or purified from comestibles. A nutraceutical is demonstrated to have a physiological benefit or provide protection against physiological disorder or discomfort. The terms “Food” or “Dietary supplement” refer to a product that contains a vitamin, mineral, herb or other botanical, amino acid, concentrate, metabolite, constituent, extract, or combinations of these ingredients. The term “Functional food” refers to any modified food or food ingredient that may provide a benefit or provide protection against physiological disorder or discomfort; beyond the traditional nutrients it contains.

As used herein, the expression “Nutraceutically effective amount” refers to the amount of a nutraceutical composition, food or dietary supplement or functional food necessary and sufficient for providing a physiological benefit or alleviating a discomfort.

The expression “Pharmaceutically effective amount” refers to the amount of a pharmaceutical composition necessary and sufficient for slowing down or stopping the progression, aggravation, or deterioration of one or more symptoms of a disease, or condition; or alleviating the symptoms of a disease or condition; or curing the disease or condition.

As used herein, the term “Transmucosal administration” refers to the administration of a product through or across a mucosal tissue. In the meaning of this invention preferred mucosal tissues are buccal, sublingual, gingival and palatal tissues. According to the invention, very preferred transmucosal administration is buccal administration. In this case, the administered substance reaches the systemic circulation through or across the mucosal tissues of the buccal cavity but is not ingested or swallowed. By “Oral administration” is meant the administration of a product in the mouth cavity followed by the swallowing of the product. In this case, the substance reaches the systemic circulation through a digestive absorption.

As used herein, an “Orodispersible/orodisintegrating tablet or film” is a tablet or a film for oral administration, whose oral administration results in its dispersion or disintegration within the mouth. As used herein, a “Chewing gum” refers to a type of gum to be chewed formed in many different shapes and sizes (balls, sticks . . . ) and made of a base comprising a natural latex product or synthetic rubber, to which are incorporated food colorings, flavorings, preservatives and other additives.

The term “Oxidation” refers to a reaction in which the atoms in an element lose electrons and the valence of the element is correspondingly increased. In a biological context, oxidation is usually linked to the reaction of a substance with oxygen, leading to the production of reactive oxygen species. The term “photo-oxidation” specifically refers to a phenomenon of oxidation facilitated or provoked by light (for example sunlight). The term “Oxidant” refers to an agent that oxidizes another agent. The term “Antioxidant” refers to an agent that decreases or avoids the oxidation of other substances. An antioxidant may protect cells against the effect of reactive oxygen species. As used herein, the term “Oxidative stress” means the imbalance between the production of reactive oxygen species in one hand and in the other hand the detoxification of the reactive intermediates and/or the repair of the resulting damages.

The expression “Photo-receptor overstimulation” refers to an excessive stimulation of the photoreceptor, which could lead to physiological problems.

As used herein, the term “Comfort” refers to the feeling of ease or well-being. Accordingly, the term “discomfort” refers to the absence or to a decrease in the feeling of ease or well-being. In one embodiment, a discomfort may be related to the presence of pain.

In one embodiment, the term “discomfort” may correspond to a loss of visual function, preferably a loss a visual function wherein the visual function becomes incompatible with driving (the requirements in term of visual function for driving are defined in the European Commission directive 2009/113/EC of 25 Aug. 2009). As used herein, the expression “visual function” may encompass one or more of the following features: visual acuity, field of vision, twilight vision, glare and contrast sensitivity and diplopia.

Methods for measuring visual function are known from the skilled artisan. For example, visual acuity may be measured by ETDRS acuity testing (ETDRS stands for Early Treatment Diabetic Retinopathy Study); field of vision may be measured by perimetry or by the use of a campimeter; glare may be measured by having the subject adjust brightness of a light source until it reaches an ill-defined threshold of unpleasantness . . . .

In one embodiment, the term “discomfort” may correspond to symptoms of eye fatigue, which may easily be measured. Examples symptoms of eye fatigue include, but are not limited to, itching, pain, winking, enhanced tear production and the like.

In a first embodiment of the invention, the visual discomfort alleviated in the present invention is non-pathological, i.e. is not related to an ocular disease, disorder or condition. According to this embodiment, alleviating a visual discomfort is a non-therapeutic action.

In a second embodiment of the invention, the visual discomfort is pathological, i.e. is the symptom of an ocular disease, disorder or condition. Examples of ocular disease, disorder or condition include, but are not limited to age related macular degeneration (AMD), diabetic retinopathy, cataract and retinitis pigmentosa. According to this embodiment, alleviating a visual discomfort is a therapeutic action and means treating a symptom of a disease, wherein the term “treating” also encompass a preventive action.

As used herein, the term “Vigilance” term encompasses one or more of the following traits: wakefulness, alertness, attentiveness, concentration and focus. Accordingly, the term “hypovigilance” refers to a deficit in one or more traits of vigilance. The state of hypovigilance may also represent an intermediate state between waking and sleeping. As used herein, an “agent for enhancing vigilance” enhances one's ability to increase and/or maintain concentration, along with the ability to ignore surrounding signals not of relevance to the situation.

In one embodiment of the invention, an agent for enhancing vigilance increases beta-1 brainwaves. Beta waves are associated with mental activity, alertness, problem solving, judgment, decision making and processing information, all components of “vigilance”. According to this embodiment, vigilance may be assessed by methods including, but not limited to, neuron-imaging techniques, such as, for example, Electro Encephalograms and Event Related Potentials, as described in WO2010/094761, which is incorporated herein by reference.

In one embodiment of the invention, the vigilance of a subject may be assessed by a questionnaire, preferably for measuring the fatigue of said subject. In one embodiment, the questionnaire may include questions about the perception of the subject regarding its capacity of concentration, its general fatigue and/or its ocular fatigue.

### DETAILED DESCRIPTION

The present invention thus relates to a composition comprising a natural extract containing anthocyanins and an agent for enhancing vigilance, said composition being in a form adapted to transmucosal administration, preferably to buccal administration, said form preferably being a chewing gum, an orodispersible/orodisintegrating tablet or film or a buccal spray. The transmucosal administration presents the advantage of an enhanced and prolonged absorption of the active ingredients of the composition, such as, for example, anthocyanins, lutein and taurin and thus improves the bioavailability of these ingredients (see Examples). In one embodiment of the invention, the composition consists of a natural extract containing anthocyanins and an agent for enhancing vigilance. Optionally, the composition of the invention comprises at least one of lutein, zeaxanthin, meso-zeaxanthin and astaxanthin.

In a first embodiment of the invention, the composition is a nutraceutical composition. In a second embodiment of the invention, the composition is a food or dietary supplement. In a third embodiment of the invention, the composition is a functional food.

In one embodiment of the invention, the composition is a pharmaceutical composition and further comprises at least one pharmaceutically acceptable excipient.

In one embodiment of the invention, the composition is in the form of an unitary dosage adapted to transmucosal administration. Examples of unitary dosages include, but are not limited to, a chewing gum or an orodispersible/orodisintegrating tablet or film or a buccal spray.

According to one embodiment of the invention, the method is such that a daily amount ranging from 0.01 to 600 mg of natural extract containing anthocyanins, preferably from 0.1 to 50 mg, more preferably from 0.5 to 20 mg may be administered to the subject. In another embodiment, the composition is such that a daily amount ranging from 0.01 to 600 mg of natural extract containing anthocyanins, preferably from 1 to 160 mg, more preferably from 10 to 100 mg may be administered to the subject. In one embodiment of the invention, said natural extract is standardized to at least 25% in weight in anthocyanins titrated on delphinidol.

In one embodiment of the invention, a unitary dosage of the composition comprises an amount of natural extract containing anthocyanins ranging from about 0.1 to about 600 mg, preferably from about 0.1 to about 200 mg, more preferably from about 0.5 to about 25 mg, even more preferably from about 1 to 15 mg, and still even more preferably from about 1.25 to about 2.5 mg of natural extract containing anthocyanins. In another embodiment of the invention, a unitary dosage of the composition comprises an amount of natural extract containing anthocyanins ranging from about 0.1 to about 50 mg, preferably from about 0.5 to 10 mg, more preferably from about 1 to 5 mg.

In one embodiment of the invention, a unitary dosage of the composition comprises an amount of natural extract containing anthocyanins ranging from about 1 to about 50% in weight to the total weight of active ingredients in the composition, preferably from about 5 to about 30% w/w, and more preferably from about 7% to about 25% w/w.

In a preferred embodiment of the invention, said natural extract containing anthocyanins is a vegetal extract.

In one embodiment of the invention, said natural extract containing anthocyanins is selected from the group comprising, but not limited to, Vaccinium Myrtillus (bilberries i.e. “bog whortleberries”), Rubus spp (blackberry i.e. “boysenberries” from north America), Rubus occidentalis (black raspberry from north America), Vaccinium corymbosum (blueberry), Vaccinium macrocarpon (American cranberry), Vaccinium oxycoccus (European cranberry), Rubus idaeus (European cranberry), Fragaria X ananassa (European cranberry), Prunus virginiana (berry i.e. “chokecherry” from North American tribal communities), Viburnum trilobum (berry i.e. “highbush cranberry” from North American tribal communities), Amelanchier alnifolia (berry i.e. “serviceberry” from North American tribal communities), Shepherdia argentea (berry i.e. “silver buffaloberry” from North American tribal communities), Rubus articus (arctic bramble), Ribes nigrum (black currant), Rubus chamaemorus (cloudberries), Empetrum nigrum, Empetrum hermaphroditum (crowberries), Sambucus spp. (elderberries), Ribes uva-crispa (gooseberry), Vaccinium vitis-idaea (lingonberries), Rubus loganobaccus (loganberry), Sorbus spp (Rowan berries), Hippophae rhamnoides (sea buckthorn), Punica granatum (pomegranate), Lycium barbarum (goji berries), Garcinia mangostana (mangosteen), Euterpe oleraceae (Brazilian acaí berry), Aristotelia chilensis (Chilean maqui berry).

In one embodiment of the invention, said natural extract containing anthocyanins is not one of Rubus occidentalis, Prunus virginiana, Ribes nigrum, Sambucus spp., Aronia melanocarpa, Aronia arbutifolia, Aronia prunifolia, Crataegus oxycantha (hawthorne berry extract), Rosemary or schisandra berry.

In a preferred embodiment of the invention, said natural extract is a gooseberry extract, preferably said gooseberry belongs to the Grossulariaceae family, gender Ribes.

In a more preferred embodiment of the invention, said natural extract containing anthocyanins is a bilberry extract. Even more preferably, said bilberry belongs to the Ericaceae family, gender Vaccinium. Even more preferably, said bilberry is Vaccinium myrtillus.

Bilberry extracts usually comprise about 25% (w/w) in anthocyanins and are commercially available, e.g. from Naturex SA (Avignon, France) or from Indena (Milan, Italy). In one embodiment, bilberry extract is from NATUREX (“Extrait de myrtille 25%”, Naturex SA, Avignon, France).

In one embodiment, the composition of the invention further comprises lutein, zeaxanthin, astanxanthin and/or meso-zeaxanthin.

In a preferred embodiment, the composition of the invention comprises lutein and zeaxanthin.

In one embodiment, the composition of the invention comprises lutein and one, two or three of lutein, zeaxanthin, astanxanthin and/or meso-zeaxanthin.

In one embodiment of the invention, the composition comprises lutein, zeaxanthin, meso-zeaxanthin and astaxanthin.

Lutein and zeaxanthin are macular pigments representing 99% of total pigments of the macula, which belong to the family of carotenoids. Lutein and zeaxanthin may exhibit on one hand an indirect antioxidant effect through their capacity to absorb blue light particularly aggressive for photoreceptor and on the other hand direct antioxidant properties. Scientific studies have shown that lutein consummation may improve eye-sensitivity to contrasts, and decreases glares.

Meso-zeaxanthin is a stereo-isomer of zeaxanthin, synthesized in the eye from lutein. It might be more efficient than lutein for essential activities in the center of the macula.

Astaxanthin is a carotenoid antioxidant with photo-protective activities. Its antioxidant properties may be more efficient than those of zeaxanthin or beta-carotene. Scientific results demonstrated that astaxanthin could be useful for preventing and treating neuronal lesions in AMD (U.S. Pat. No. 5,527,533).

In one embodiment, the method of the invention is such that a daily amount ranging from 0.01 to 12 mg of lutein and zeaxanthin, preferably from 0.05 to 6 mg, more preferably from 0.1 to 1.2 mg may be administered to the subject.

In one embodiment of the invention, a unitary dosage of the composition comprises an amount of lutein and zeaxanthin ranging from about 0.01 to 12 mg, preferably from about 0.02 to 6 mg, more preferably from about 0.05 to 2 mg.

In one embodiment, the method of the invention is such that a daily amount ranging from about 0.01 to 50 mg of lutein, preferably from about 0.01 to 10 mg of lutein, more preferably from 0.05 to 5 mg, even more preferably from 0.1 to 1.0 mg may be administered to the subject.

In one embodiment of the invention, a unitary dosage of the composition comprises an amount of lutein ranging from about 0.01 to 50 mg, preferably from about 0.01 to 12 mg, more preferably from about 0.05 to 10 mg, even more preferably from about 0.1 to 5 mg of lutein.

In one embodiment of the invention, a unitary dosage of the composition comprises an amount of lutein ranging from about 0.1 to about 50% in weight to the total weight of active ingredients in the composition, preferably from about 0.5 to about 40% w/w, more preferably from about 0.85% to about 30% w/w.

In one embodiment, the method of the invention is such that a daily amount ranging from 0.001 to 12 mg of zeaxanthin, preferably from about 0.001 to 2 mg, more preferably from 0.005 to 1 mg, even more preferably from 0.01 to 0.2 mg may be administered to the subject.

In one embodiment of the invention, a unitary dosage of the composition comprises an amount of zeaxanthin ranging from about 0.001 to about 12 mg, preferably from about 0.001 to about 2.4 mg, more preferably from about 0.01 to about 1 mg, even more preferably from about 0.02 to 0.5 mg of lutein.

In one embodiment of the invention, a unitary dosage of the composition comprises an amount of zeaxanthin ranging from about 0.01 to about 10% in weight to the total weight of active ingredients in the composition, preferably from about 0.05 to about 5% w/w, more preferably from about 0.15% to about 3% w/w.

In one embodiment, lutein and zeaxanthin are present in a 10/1 to 5/1 ratio. In one embodiment, lutein and zeaxanthin are present in a 10/1 ratio. In another embodiment, lutein and zeaxanthin are present in a 5/1 ratio.

Purified lutein is commercially available (FloraGlo® from FluraGloLutein).

In an embodiment of the invention, lutein is partially or totally substituted by meso-zeaxanthin, astaxanthin or a mix thereof.

In an embodiment of the invention, zeaxanthin is partially or totally substituted by meso-zeaxanthin, astaxanthin or a mix thereof.

In one embodiment of the invention, said agent for enhancing vigilance is at least one of caffeine, taurin, Vitamine C, theanine and oregano, preferably at least one of caffeine, taurin and vitamin C, more preferably said agent for enhancing vigilance is caffeine.

In one embodiment of the invention, said agent for enhancing vigilance is caffeine. Caffeine acts as a stimulating agent of the central nervous system and of cardiovascular system. Caffeine may be used for reducing physical fatigue, for enhancing attention in order to avoid slumber and for insuring a good corporal coordination.

In a preferred embodiment, the composition of the invention includes caffeine, either from a natural extract such as for example guarana or not from a natural extract (i.e. synthetic caffeine), or both.

In one embodiment of the invention, the method is such that a daily amount ranging from 0.1 to 200 mg of caffeine, preferably from 1 to 150 mg, more preferably from 10 to 100 mg may be administered to the subject.

In one embodiment of the invention, a unitary dosage of the composition comprises an amount of caffeine ranging from about 0.1 to about 200 mg, preferably from about 1 to about 100 mg, preferably from about 2.5 to about 20 mg, more preferably from about 5 to 11 mg of caffeine. In one embodiment of the invention, a unitary dosage of the composition comprises an amount of caffeine ranging from about 1 to about 150 mg, preferably from about 2 to about 50 mg, more preferably from about 5 to about 20 mg.

In one embodiment of the invention, a unitary dosage of the composition comprises an amount of caffeine ranging from about 40 to about 95% in weight to the total weight of active ingredients in the composition, preferably from about 45 to about 90% w/w, more preferably from about 49% to about 88% w/w.

In one embodiment of the invention, the composition of the invention further comprises Vitamin C. Vitamin C is a non-enzymatic water-soluble antioxidant. Vitamin C may act as an antioxidant agent in the eye. Vitamin C may also help to counteract the risk of deterioration of the visual functions (AREDS reports n° 8, Arch Ophtalmol, 2001; 119:1417-36).

In an embodiment, the method of the invention is such that a daily amount ranging from 0.1 to 1000 mg of Vitamin C, preferably from 1 to 300 mg, more preferably from 10 to 280 mg, even more preferably about 240 mg may be administered to the subject. In another embodiment, the composition is such that a daily amount ranging from 0.1 to 240 mg of Vitamin C may be administered to the subject. In another embodiment, the composition is such that a daily amount ranging from about 0.1 to about 60 mg, preferably about 60 mg of Vitamin C may be administered to the subject.

In one embodiment of the invention, a unitary dosage of the composition comprises an amount of vitamin C ranging from about 0.01 to about 1000 mg, preferably from about 0.01 to about 240 mg, more preferably from about 0.1 to about 60 mg, and even more preferably from about 1 to 30 mg of vitamin C. In one embodiment of the invention, a unitary dosage of the composition comprises an amount of vitamin C ranging from about 0 to about 200 mg. In one embodiment of the invention, a unitary dosage of the composition comprises an amount of vitamin C ranging from about 0 to about 60 mg.

According to the present invention, ascorbic acid, sodium ascorbate, calcium ascorbate, potassium ascorbate, ascorbyl palmitate, magnesium ascorbate, zinc ascorbate or a mix thereof can be present in the composition of the invention, as a source of vitamin C, preferably ascorbic acid, sodium ascorbate or a mix thereof. Preferably, ascorbic acid is used as the source of vitamin C.

In one embodiment, the composition of the invention further comprises taurin.

Taurin (aminoethanesulfonic acid) may play a major role in development and survey of retinal neurons. Taurin may also protect retinal cells in case of diabetic retinopathies or photo-induced stress through antioxidant properties (Yu et al., Neurochem Res 2008; 33(3):500-7; Yu et al., Br J Nutr 2007; 98(4):711-9). Deficit in taurin may cause deprotection of retina from photo-oxidation resulting in loss of visual acuity.

In one embodiment of the invention, the method of the invention is such that a daily amount ranging from 0.1 to 200 mg of taurin, preferably from 1 to 100 mg, more preferably from 10 to 50 mg may be administered to the subject.

In one embodiment of the invention, a unitary dosage of the composition comprises an amount of taurin ranging from about 0.1 to about 200 mg, preferably from about 0.01 to about 50 mg, more preferably from about 0.1 to about 40 mg, and even more preferably from about 1 to 30 mg of taurin. In one embodiment of the invention, a unitary dosage of the composition comprises an amount of taurin ranging from about 0 to about 200 mg.

In a preferred embodiment, highly purified taurin (minimum 98.5%) is used.

In one embodiment of the invention, the composition of the invention comprises a natural extract containing anthocyanins, caffeine, lutein and zeaxanthin. In another embodiment of the invention, the composition of the invention consists of natural extract containing anthocyanins, caffeine, lutein and zeaxanthin.

In one embodiment of the invention, the composition of the invention is in the form of a chewing gum comprising a natural extract containing anthocyanins and an agent for enhancing vigilance comprising:
1.25 to 2.5 mg of natural extract containing anthocyanins,
5 to 11 mg of caffeine,
0.1 to 5 mg of lutein,
0.02 to 0.5 mg of zeaxanthin.

The present invention also relates to a chewing gum or to an orodispersible/orodisintegrating tablet or film comprising the composition of the invention. In one embodiment, due to the size of such products, the quantity of active components is limited to 10 to 1000 mg, preferably to 50 to 540 mg per gum or per tablet or per film. In one embodiment of the invention, the quantity of active components is limited to 50 mg per unitary dosage. In one embodiment of the invention, the quantity of active components is limited to 540 mg per unitary dosage.

In one embodiment, the amount of active ingredient in the chewing gum or in the orodispersible/orodisintegrating tablet of the invention ranges from about 1 to about 50 mg, preferably about 5 to about 30 mg, more preferably from about 10 to about 20 mg.

In one embodiment, the active ingredients include anthocyanin, lutein, zeaxanthin, caffeine, taurin, vitamin C, oregano, theanine, and the like.

In one embodiment of the invention, the chewing gum or the orodispersible/orodisintegrating tablet or film comprises from about 0.1 to about 10% of active ingredient in weight to the weight of the total chewing gum or orodispersible/orodisintegrating tablet, preferably from about 0.5 to about 5% w/w of active ingredients, more preferably from about 0.75 to about 1.5% w/w of active ingredients.

In another embodiment of the invention, the chewing gum or the orodispersible/orodisintegrating tablet or film comprises from about 0.1 to about 95% of active ingredient in weight to the weight of the total chewing gum or orodispersible/orodisintegrating tablet, preferably from about 50 to about 90% w/w of active ingredients, more preferably from about 85 to about 90% w/w of active ingredients.

The present invention also relates to a composition comprising a natural extract containing anthocyanins and an agent for enhancing vigilance for alleviating or for use in alleviating visual discomfort, said composition being in a form adapted to transmucosal administration, said form preferably being a chewing gum, an orodispersible/orodisintegrating tablet or film or a buccal spray. Said composition consisting of a natural extract containing anthocyanins and an agent for enhancing vigilance may be a nutraceutical composition, a food or dietary supplement, a functional food or a pharmaceutical composition. In one embodiment, the composition for use in alleviating visual discomfort as hereinabove described consists of a natural extract containing anthocyanins and an agent for enhancing vigilance.

The present invention also relates to the use of the composition as hereinabove described for alleviating a visual discomfort, preferably related to or caused by oxidative stress, more preferably due to photo-oxidation or photoreceptor overstimulation, especially of the retina in a subject in need thereof. Examples of visual discomforts include, but are not limited to, a poor night and twilight vision, an enhanced light sensitivity, a poor visual acuity, a narrowed field of vision, glare, contrast sensitivity, diplopia and symptoms of eye fatigue.

The present invention also relates to the use of the composition as hereinabove described for enhancing vigilance in a subject.

The present invention also relates to the use of the composition as hereinabove described for decreasing reaction time in a subject. The reaction time of a subject may depend on visual factors, such as, for example, the capacity to promptly recover normal vision after glare, and/or on the vigilance state of the subject.

The present invention also relates to the use of the composition as hereinabove described for enhancing visual function, for enhancing vigilance and for decreasing reaction time in a subject. According to this embodiment, the composition of the invention is of particular interest for drivers, preferably for car drivers, as it may reduce the risk of car crash due to human factors.

According to an embodiment, the composition of the invention has a court-term action, a mid-term action and a long-term action.

In one embodiment of the invention, the composition of the invention is for alleviating a visual discomfort symptomatic of an ocular disease in a subject in need thereof. Preferably, said ocular disease is a disease of the photoreceptors of the eyes. Examples of diseases of the photoreceptors of the eye include, but are not limited to macular dystrophies, cone-rod dystrophies, enhanced eye-cone syndrome, inherited severe photoreceptor diseases, such as, for example, retinitis pigmentosa and leber congenital amaurosis, human hereditary retinopathies, retinal degeneration, retinal degenerative diseases, red-cone degeneration, stargardt disease, fundus flavimaculatus, photoreceptor dysplasia and progressive retinal atrophy.

In one embodiment, said subject affected by an ocular disease resulting in visual discomfort is simultaneously treated with a pharmaceutical treatment for said disease.

The present invention also relates to a method for alleviating visual discomfort in a subject in need thereof, preferably a visual discomfort related to or caused by oxidative stress, comprising administering to said subject a nutraceutically or pharmaceutically effective amount of the composition of the invention.

In one embodiment of the invention, said visual discomfort related to or caused by oxidative stress is due to photo-oxidation or photoreceptor overstimulation, especially of the retina. Examples of visual discomfort include, but are not limited to, a poor night and twilight vision, an enhanced light sensitivity, a poor visual acuity, a narrowed field of vision, glare, contrast sensitivity, diplopia and symptoms of eye fatigue.

Another object of the invention is a method for enhancing visual function, for enhancing vigilance and/or for decreasing reaction time in a subject comprising administering to said subject a nutraceutically or pharmaceutically effective amount of the composition of the invention.

Another object of the invention is a method for alleviating a visual discomfort, for enhancing visual function, for enhancing vigilance and/or for decreasing reaction time in a subject in need thereof comprising administering to said subject, preferably by a transmucosal route, a daily amount:
of natural extract containing anthocyanins ranging from 0.01 to 600 mg,
of lutein ranging from 0 to 10 mg, preferably from 0.01 to 10 mg,
of zeaxanthin ranging from 0 to 2 mg, preferably from 0.001 to 2 mg,
of caffeine ranging from 0.1 to 200 mg,
of vitamin C ranging from 0 to 1000 mg, preferably from 0 to 240 mg,
of taurin ranging from 0 to 200 mg.

According to an embodiment, the composition of the invention is administered in an amount such that the subject intakes the following daily amounts:
of natural extract containing anthocyanins ranging from 0.01 to 600 mg,
of lutein ranging from 0 to 10 mg, preferably from 0.01 to 10 mg,
of zeaxanthin ranging from 0 to 2 mg, preferably from 0.001 to 2 mg,
of caffeine ranging from 0.1 to 200 mg,
of vitamin C ranging from 0 to 1000 mg, preferably from 0 to 240 mg,
of taurin ranging from 0 to 200 mg.

The recommended daily consummation according to the invention ranges from 1 to 20 chewing gums or from 1 to 2 orodispersible/orodisintegrating tablets or films. The daily intake is limited by the maximum daily amount for each ingredient.

According to an embodiment, the composition of the invention is recommended to a subject exposed to fatigue or to environmental stress, such as, for example, tobacco, air pollution, UV light and the like causing oxidative stress to the eye.

According to an embodiment, the subject is more than 40, preferably more than 50, more preferably more than 60 years old.

The composition of the invention may be a visual care product, which means that it maintains visual comfort.

In one embodiment of the invention, the subject is a driver, such as, for example, a car driver.

According to an embodiment, the subject receiving the composition of the invention is a mammal, such as, for example, a pet or a primate, preferably a human.

### BRIEF DESCRIPTION OF THE DRAWINGS

FIG. 1 is a graph showing comparative data of anthocyanin absorption, with regard to the administration mode and anthocyanin amount.

### EXAMPLES

The present invention is further illustrated by the following examples.

### Example 1

### Composition of the Invention

The bilberry extract used in the following examples of composition are from Naturex (Naturex SA, Avignon, France) and contained 25% in weight of anthocyanins to the total weight of the extract. FloraGlo® is from FloraGloLutein (KEMIN Health, 600E, Court Ave, Des Moines, USA) and contained Lutein/Zeaxanthin in a 5/1 ratio. It is used in the present invention as a source of both lutein and zeaxanthin. Caffeine is from Naturex.

| | Composition number | | | | | | | | | | |
|---|---|---|---|---|---|---|---|---|---|---|---|
| Nutriment | 1 | 2 | 3 | 4 | 5 | 6 | 7 | 8 | 9 | 10 | 11 |
| Bilberry extract | 12.5 | 12.5 | 12.5 | 12.5 | 12.5 | 12.5 | 12.5 | 12.5 | 12.5 | 12.5 | 12.5 |
| Caffeine | 100 | 0 | 0 | 0 | 100 | 0 | 0 | 100 | 0 | 100 | 100 |
| Vitamin C | 0 | 240 | 0 | 0 | 0 | 240 | 240 | 240 | 240 | 240 | 240 |
| Taurin | 0 | 0 | 0 | 10 | 0 | 0 | 10 | 0 | 10 | 0 | 10 |
| Lutein/Zeaxanthin | 1.2 | 0 | 1.2 | 0 | 0 | 1.2 | 0 | 0 | 1.2 | 1.2 | 1.2 |

The amounts are indicated in mg per daily administration.

### Example 2

### Chewing Gums

Composition in weight (mg) of three chewing gums of the invention.

| | | Weight (mg) | | |
|---|---|---|---|---|
| | | Chewing | Chewing | Chewing |
| Ingredients | Function | gum 1 | gum 2 | gum 3 |
| Xylitol | Sweetener | 367.20 | 401.08 | 367.05 |
| Sorbitol | Sweetener | 356.40 | 349.68 | 390.20 |
| Gum base | gum | 285.88 | 254.92 | 254.90 |
| Mannitol | Sweetener | 72 | 71.98 | 71.97 |
| Sirup of maltitol | Sweetener | 68 | 67.98 | 67.97 |
| Arum menthol | Aroma | 33.75 | 33.74 | 33.74 |
| Arum mint | Aroma | 21 | 20.99 | 20.99 |
| Arabic gum | Thickening agent | 12 | 12 | 12 |
| lecithine | Emulsifier | 8.50 | 8.50 | 8.50 |
| Dioxide titanium | colouring agent | 8 | 8 | 8 |
| Sucralose | Sweetener | — | 1.36 | 1.36 |
| Acesulfam K | Sweetener | 2.55 | — | — |
| Mint | Aroma | 2.50 | 2.50 | 2.50 |
| Aspartame | Sweetener | 0.85 | — | — |
| Carnauba wax | Thickening agent | — | 0.38 | 0.51 |
| Bilberry extract | Active ingredient | 1.25 | 1.25 | 2.50 |
| Caffeine | Active ingredient | 10 | 10.15 | 5.08 |
| Lutein | Active ingredient | 0.1 | 5 | 2.50 |
| Zeaxanthin | Active ingredient | 0.02 | 0.50 | 0.25 |
| TOTAL | | 1250 | 1250 | 1250 |

Composition of active ingredient of three chewing gums of the invention (% in weight to the weight of the total active ingredients)

| | % (w/w) | | |
|---|---|---|---|
| Ingredients | Chewing gum 1 | Chewing gum 2 | Chewing gum 3 |
| Bilberry extract | 10.99 | 7.40 | 24.20 |
| Caffeine | 87.95 | 60.05 | 49.18 |
| Lutein | 0.88 | 29.59 | 24.20 |
| Zeaxanthin | 0.18 | 2.96 | 2.42 |
| TOTAL | 100 | 100 | 100 |

Composition of excipients and active ingredient of three chewing gums of the invention in weight to the weight of the chewing gum

| | % (w/w) | | |
|---|---|---|---|
| Ingredients | Chewing gum 1 | Chewing gum 2 | Chewing gum 3 |
| Excipients | 99.09 | 98.65 | 99.17 |
| Active ingredients | 0.91 | 1.35 | 0.83 |
| TOTAL | 100 | 100 | 100 |

### Example 3

### Orodispersible/Orodisintegrating Tablet

12.5 mg of Bilberry extract
100 mg of caffeine
1 mg of lutein
0.2 mg of zeaxanthin

### Example 4

### Material

### Rats OFA, Sprague-Dawlay

The stick gum administered to Rats contains 5 mg of vegetal extract titrated at 25% of anthocyanosides (i.e. 1.250 μg of anthocyanosides).

Trypsin and collagenase are from Worthington Biochemical Corporation, Lakewood, N.J.

### Method

### Diets

The Sprague Dawley rat (10 Males) used extensively in medical research weighing 450-520 g were received and placed separately in cage in room temperature controlled (22° C.). This rat species is chose for its calmness and its easy handling. Rats were maintained in dark periode condition and fed a diet (755 g/kg wheat starch, 150 g/kg casein, 50 g/kg peanut oil, 35 g/kg AlN-93M mineral mixture, 10 g/kg AlN-76A vitamin mixture) during 2 weeks (Felgines, C. et al. J. Nutr 2002).

### Anthocyanin Stability and Detection

As anthocyanins are particularly susceptible to pH variations and are unstable at intestinal pH, collected samples were analyzed by HPLC after acidification with 240 mmol/L HCl, to allow the comparison with intestinal administration. Plasmatic anthocyanins levels were assessed after plasma separation of blood samples by centrifugation and extracted with a solid-phase extraction cartridge (Sep-Pak C18 Plus) and then analyzed by HPLC possessing a photodiode array detector and an UV-visible detector (785A, Perkin-Elmer) at 524 nm. Spectrum was compared to Cyanidin 3-glucoside that represent 98% of total anthocyanins. Anthocyanin metabolite analysis was carried out by HPLC-electrospray ionization (ESI)-MS-MS analysis and performed on a HPLC system equipped with MS-MS detection (API 2000). Anthocyanin metabolites were detected according to the respective m/z values of their parent and product ions.

Absorption from mucosal epithelium and serum was estimated by calculating the difference between anthocyanin quantity contained in buccal gum patch administered and the amount recovered at the end in samples.

### Buccal Administration Process

Rats (OFA, Sprague-Dawlay) received a lingual administration of anthocyanins with an orodispersible gum containing 1.25 mg of anthocyanin. The stick gum was deposited at the buccal mucosa area.

Peripheral blood was collected 1 hour post-administration, centrifuged and plasma was acidified through formic acid addition. After acidification, plasma samples were conserved at −80° C. and analyzed by LC-MS/MS (Liquid Chromatography—Mass Spectrometer/Mass Spectrometer).

In parallel, epithelium and conjunctive tissues were collected 45 minutes post-administration, placed in formic acid and eventually conserved at −80° C. Before LC-MS/MS analyze, tissues were washed in PBS (phosphate buffered saline) and homogenized on ice. Protein concentrations were determined by Lowry method. To facilitate tissues preparation, samples were homogenized and treated with 2.5% trypsin and 2% collagenase during 1.5 h at 37° C., and then acidified with formic acid.

Treatment with formic acid does not affect anthocyanins stability.

Absorption was estimated by calculating the administering/recovering anthocyanosides estimated concentration in samples ratio. Quantification was adjusted in respect of residual anthocyanoside quantity contained in buccal washing (anthocyanosides extracted from stick gum not absorbed and residual non dissolved stick gum).

### Digestive Administration Process

Digestive administration of anthocyanosides is done as previously described (S Talayera, C Felgines, O Texier et al. J. Nutr. 134: 2275-2279, 2004).

Briefly, samples were collected at the beginning and after perfusion period (45 min), and anthocyanins were analyzed by HPLC as previously described. Intestinal perfusion method (Crespy, V., Morand, C., Manach, C., et al. Am. J. Physiol. 277: G120-G126, 1999) is done using bile duct cannulation consist in jejunal plus intestine ileal segment (flexura duodenojejunalis to the valvula ileocecalis) perfusion. This segment was perfused for 45 min at a flow rate of 0.75 mL/min with a physiological buffer containing KH₂PO₄ (5 mmol/L), K₂HPO₄ (2.5 mmol/L), NaHCO₃ (5 mmol/L), NaCl (50 mmol/L), KCl (40 mmol/L), CaCl₂ (2 mmol/L), MgSO₄ (1 mmol/L), tri-potassium citrate (10 mmol/L), glucose (12 mmol/L), glutamine (2 mmol/L), and taurocholic acid (1 mmol/L), pH 6.6, at 37° C. supplemented with blackberry extract containing 25% and 50%, respectively. Absorption was estimated by calculating the administrating/recovering anthocyanins ratio.

Extract containing 25% of Anthocyanins is purchased from Naturex SA (Avignon, France). Extract containing 50% of Anthocyanins is purchased from Ferlux Mediolanum SA (Cournon d'Auvergne, France).

Anthocyanosides concentration is based on Delphinidine (C₁₅H₁₁ClO₇, PM=338.70 g.mol⁻¹).

### REFERENCES

Felgines, C., et al. (2002). Blackberry anthocyanins are slightly bioavailable in rats. J. Nutr. 132: 1249-1253
Talayera S., et al. (2004). Anthocyanins are efficiently absorbed from the small intestine in rats. J. Nutr. 134: 2275-2279
Crespy, V., et al. (1999). Part of quercetin absorbed in the small intestine is conjugated and further secreted in the intestinal lumen. Am. J. Physiol. 277: G120-G126

### Results

### Buccal Absorption Following Lingual Administration of the Stick Gum of the Invention:

LC-MS/MS results, corresponding to the absorption of anthocyanin are presented on Table 1. Table 1 presents the amount of anthocyanin in the buccal tissue, and the amount of anthocyanin in the plasma.

TABLE 1

| | Anthocyanin | Anthocyanin |
|---|---|---|
| RATS | (ng/mg of | (μg/ml of |
| (n^{o}) | buccal tissue) | plasma) |
| 1. | 700 | 200 |
| 2. | 701 | 210 |
| 3. | 745 | 235 |
| 4. | 755 | 243 |
| 5. | 699 | 227 |
| 6. | 735 | 190 |
| 7. | 780 | 207 |
| 8. | 810 | 220 |
| 9. | 785 | 245 |
| 10. | 785 | 150 |
| Mean +/− | 749 +/− 12 | 212 +/− 9 |
| SEM | | |

### Comparative Digestive Absorption Following Intestinal Perfusion:

LC-MS/MS results, corresponding to the absorption of anthocyanin are presented on table 2. Table 2 presents the amount of anthocyanin in the tissues and in the plasma.

TABLE 2

| | Anthocyanin | | Anthocyanin | |
|---|---|---|---|---|
| | (nanogramme/mg of tissue) | | (microgramme/ml of plasma) | |
| Rats | Anthocyanin | Anthocyanin | Anthocyanin | Anthocyanin |
| (n^{o}) | 25% | 50% | 25% | 50% |
| 1. | 20.0 | 380.0 | 5.7 | 108.6 |
| 2. | 20.0 | 380.5 | 6.0 | 114.0 |
| 3. | 21.3 | 404.4 | 6.7 | 127.6 |
| 4. | 21.6 | 409.9 | 6.9 | 131.9 |
| 5. | 20.0 | 379.5 | 6.5 | 123.2 |
| 6. | 21.0 | 399.0 | 5.4 | 103.1 |
| 7. | 22.3 | 423.4 | 5.9 | 112.4 |
| 8. | 23.1 | 439.7 | 6.3 | 119.4 |
| 9. | 22.4 | 426.1 | 7.0 | 133.0 |
| 10. | 22.4 | 426.1 | 4.3 | 81.4 |
| Mean +/− | 21.41 +/− 0.36 | 406.87 +/− 6.94 | 6.07 +/− 0.25 | 115.46 +/− |
| SEM | | | | 4.91 |

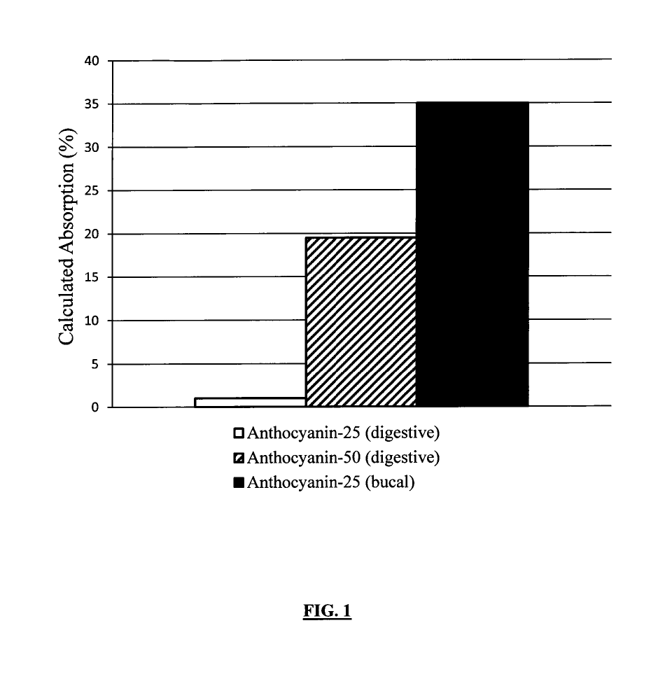

Comparison of buccal and digestive absorption of anthocyanins is presented on FIG. 1.

Anthocyanins are significantly absorbed in the tissues and in the plasma. Moreover, the absorption of anthocyanin is more effective when the administration is done through the lingual way, as compared to the digestive way.

Indeed, as shown on FIG. 1, the calculated absorption of anthocyanin is more than 10 fold superior when the vegetal extract containing anthocyanin is administered by the lingual route (compare “Anthocyanin-25 (buccal)” and “Anthocyanin-25 (digestive)”).

Moreover, the lingual route of administration allows the administration of an amount of anthocyanin lower than with a digestive route, for the same biological result. Indeed, as shown in FIG. 1, the amount of absorbed anthocyanin is greater when a vegetal extract containing anthocyanin titrated at 25% of anthocyanin is administered by a lingual route than when an extract titrated at 50% is administered by a digestive route (compare “Anthocyanin-25 (buccal)” and “Anthocyanin-50 (digestive)”).

### Example 5

### Experimental Procedure

Rats received a fed diet or gum containing Taurine (50 mg/kg or 100 mg/kg), Lutein (6 mg/kg and 12 mg/kg), and Anthocyanine (200 mg/kg and 400 mg/kg) to follow digestive versus sublingual absorption of these compounds, then blood sample are harvested 1 hour after administration. Taurin and Lutein plasmatic level are evaluated using HPLC process as described Lallemand F. et al, 2004 and Shanmugam S, et al, 2011, respectively.

### REFERENCES

Lallemand F. and De Witte Ph. Taurine concentration in the brain and in the plasma following intraperitoneal injections; Amino Acids, (2004) 26: 111-116.
Shanmugam S, Baskaran R, Balakrishnan P, Thapa P, Yong C S, Yoo B K. Solid self-nanoemulsifying drug delivery system (S—SNEDDS) containing phosphatidylcholine for enhanced bioavailability of highly lipophilic bioactive carotenoid lutein. Eur J Pharm Biopharm. 2011 Apr. 29.

### Absorption of Lutein:

Digestive versus sublingual absorption of Lutein after 1 hour of administration.

| | Plasmatic lutein concentration (ng/ml) | | |
|---|---|---|---|
| Administration route | Baseline | 6 mg/kg | 12 mg/kg |
| Digestive | 25 +/− 5 | 34 +/− 4 | 32 +/− 5 |
| Sublingual | 26 +/− 5 | 120 +/− 12 | 160 +/− 10 |

### Absorption of Taurine:

Digestive versus sublingual absorption of Taurine after 1 hour of administration.

| | Plasmatic Taurine concentration (μM) | | |
|---|---|---|---|
| Administration route | Baseline | 50 mg/kg | 125 mg/kg |
| Digestive | 301 +/− 15 | 605 +/− 18 | 623 +/− 25 |
| Sublingual | 280 +/− 9 | 1490 +/− 22 | 3020 +/− 50 |

### Absorption of Anthocyanins (Used as a Control Procedure Based on the FIG. 1)

Digestive versus sublingual absorption of Anthocyanin after 1 hour of administration.

| | Plasmatic Anthocyanin concentration (μg/ml) | | |
|---|---|---|---|
| Administration route | Baseline | 200 mg/kg | 400 mg/kg |
| Digestive | N. D. | 1.5 +/− 0.2 | 2.7 +/− 0.3 |
| Sublingual | N. D. | 45.8 +/− 1.0 | 79.7 +/− 1.4 |

N.D: not detectable

The results presented in the tables above showed that the absorption of taurine, lutein and anthocyanin is more effective when the administration is done through the lingual way, as compared to the digestive way.

### Example 6

### Effect of the Composition of the Invention on Car Drivers

The efficacy of the composition of the invention on visual function, vigilance and reaction time were assessed in subjects receiving one chewing gum of the invention.

### Method

30 subjects (age ranging from 18 to 65) have received one chewing gum of the invention, or one chewing gum without active ingredient (control).

### Visual Function Assessment

Visual function was assessed by visual acuity measurement (ETDRS acuity testing), field of vision measurement, sensibility to contrast and glare measurement, and perception of visual fatigue by the subject. For evaluating visual fatigue, the subject evaluated the frequency/duration of the symptoms of eye fatigue by giving a notation from 0 (absence) to 10 (continuous presence). Examined symptoms of eye fatigue were itching, pain, winking and enhanced tear production.

### Vigilance Assessment

Vigilance was assessed using a questionnaire. The questionnaire comprised six questions, related to the perception of the subject regarding its capacity of concentration, its general fatigue, its wakefulness, its alertness, its attentiveness, and its focus. For each question, the subject has to give a notation from 0 (very poor) to 10 (very high).

### Reaction Time Assessment

For assessing the reaction time of a subject, the following test was carried out: the subject presses the space bar every time an asterisk appears on the computer screen (the interval between appearances is random). The response time corresponds to the interval between the appearance of the asterisk and the action to press the space bar.

### Results

Reported returns were positive in the three categories: lower eye fatigue associated with lower tear production and winking; lower perception of general fatigue and personal assessment, better perception of attentiveness and alertness. Reaction time assessment was also improved.

1. A composition comprising a natural extract containing anthocyanins, an agent for enhancing vigilance, and optionally at least one of lutein, zeaxanthin, meso-zeaxanthin and asthaxanthin; said composition being in an unitary dosage form adapted to transmucosal administration, said unitary dosage form preferably being a chewing gum, an orodispersible/orodisintegrating tablet or film or a buccal spray.

2. A composition according to claim 1, wherein said composition is a nutraceutical composition, a food or dietary supplement, a functional food or a pharmaceutical composition.

3. A composition according to claim 1 or 2, wherein said transmucosal administration is a buccal administration.

4. A composition according to anyone of claims 1 to 3, wherein said agent for enhancing vigilance is at least one of caffeine, taurin and vitamin C, preferably caffeine.

5. A composition according to anyone of claims 1 to 4, wherein said unitary dosage form comprises an amount of natural extract containing anthocyanins ranging from 0.1 to 200 mg.

6. A composition according to anyone of claims 1 to 5, wherein said natural extract containing anthocyanins is a vegetal extract, said vegetal being selected from the group comprising, but not limited to, Vaccinium Myrtillus (bilberries i.e. “bog whortleberries”), Rubus spp (blackberry i.e. “boysenberries” from north America), Rubus occidentalis (black raspberry from north America), Vaccinium corymbosum (blueberry), Vaccinium macrocarpon (American cranberry), Vaccinium oxycoccus (European cranberry), Rubus idaeus (European cranberry), Fragaria X ananassa (European cranberry), Prunus virginiana (berry i.e. “chokecherry” from North American tribal communities), Viburnum trilobum (berry i.e. “highbush cranberry” from North American tribal communities), Amelanchier alnifolia (berry i.e. “serviceberry” from North American tribal communities), Shepherdia argentea (berry i.e. “silver buffaloberry” from North American tribal communities), Rubus articus (arctic bramble), Ribes nigrum (black currant), Rubus chamaemorus (cloudberries), Empetrum nigrum, Empetrum hermaphroditum (crowberries), Sambucus spp. (elderberries), Ribes uva-crispa (gooseberry), Vaccinium vitis-idaea (lingonberries), Rubus loganobaccus (loganberry), Sorbus spp (Rowan berries), Hippophae rhamnoides (sea buckthorn), Punica granatum (pomegranate), Lycium barbarum (goji berries), Garcinia mangostana (mangosteen), Euterpe oleraceae (Brazilian acaí berry), Aristotelia chilensis (Chilean maqui berry).

7. A composition according to anyone of claims 1 to 6, wherein natural extract containing anthocyanins is a bilberry extract, preferably said bilberry belongs to the Ericaceae family, gender Vaccinium, more preferably the bilberry is Vaccinium myrtillus.

8. A composition according to anyone of claims 1 to 7, wherein natural extract containing anthocyanins is a gooseberry extract, preferably said gooseberry belongs to the Grossulariaceae family, gender Ribes.

9. A composition according to anyone of claims 1 to 8, wherein said unitary dosage composition comprises an amount ranging from 1 to 100 mg of caffeine.

10. A composition according to anyone of claims 1 to 9, comprising lutein in an amount ranging from 0.01 to 50 mg and zeaxanthin in an amount ranging from 0.001 to 12 mg.

11. A composition according to anyone of claims 1 to 10, wherein the unitary dosage form further comprises one or more of the following ingredients:
Vitamin C, in an amount ranging from 0.01 to 240 mg,
Taurin, in an amount ranging from 0.01 to 50 mg.

12. A composition according to anyone of claims 1 to 11, wherein said composition is in the form of a chewing gum comprising a natural extract containing anthocyanins and an agent for enhancing vigilance comprising:
1.25 to 2.5 mg of natural extract containing anthocyanins,
5 to 11 mg of caffeine,
0.1 to 5 mg of lutein,
0.02 to 0.5 mg of zeaxanthin.

13. A composition according to anyone of claims 1 to 12, for alleviating a visual discomfort, for enhancing visual function, for enhancing vigilance and/or for decreasing reaction time in a subject in need thereof.

14. A composition according to claim 13, wherein visual discomfort is related to oxidative stress, said oxidative stress being due for example to photo-oxidation or photoreceptor overstimulation, especially of the retina.

15. A composition according to claim 13 or 14, wherein said visual discomfort is a poor night vision, an enhanced light sensitivity or glare.

16. A composition for alleviating a visual discomfort in a subject in need thereof according to anyone of claims 13 to 15, wherein the composition is administered in an amount such that the subject intakes the following daily amounts:
of natural extract containing anthocyanins ranging from 0.01 to 600 mg,
of lutein ranging from 0.01 to 10 mg,
of zeaxanthin ranging from 0.001 to 2 mg,
of caffeine ranging from 0.1 to 200 mg,
of vitamin C ranging from 0 to 1000 mg, preferably from 0 to 240 mg,
of taurin ranging from 0 to 200 mg.

17. A method for alleviating visual discomfort, for enhancing visual function, for enhancing vigilance and/or for decreasing reaction time in a subject in need thereof comprising administering to said subject a nutraceutically effective amount or a pharmaceutically effective amount of the composition according to anyone of claims 1 to 12.

18. A method for alleviating a visual discomfort, for enhancing visual function, for enhancing vigilance and/or for decreasing reaction time in a subject in need thereof comprising administering to said subject a daily amount:
of natural extract containing anthocyanins ranging from 0.01 to 600 mg,
of lutein ranging from 0.01 to 10 mg,
of zeaxanthin ranging from 0.001 to 2 mg,
of caffeine ranging from 0.1 to 200 mg,
of vitamin C ranging from 0 to 1000 mg, preferably from 0 to 240 mg,
of taurin ranging from 0 to 200 mg.
